(19) United States
(12) Patent Application Publication
(10) Pub. No.: US 2024/0307057 A1
(43) Pub. Date: Sep. 19, 2024

(54) NON-CUTTING BARIATRIC COUPLING DEVICE AND METHOD OF USE

(71) Applicant: W. L. Gore & Associates, Inc., Newark, DE (US)

(72) Inventor: David J. Lane, West Grove, PA (US)

(21) Appl. No.: 18/571,152

(22) PCT Filed: Jun. 10, 2022
(86) PCT No.: PCT/US2022/032968

§ 371 Date: Dec. 15, 2023

Related U.S. Application Data
(60) Provisional application No. 63/213,364, filed on Jun. 22, 2021.

(51) Int. Cl. A61B 17/064; A61B 17/00; A61B 17/04; A61B 17/072

(52) U.S. Cl. CPC A61B 17/0643; A61B 17/07207; A61B 2017/00818; A61B 2017/00876; A61B 2017/0408; A61B 2017/0409

(57) ABSTRACT

An endoscopic gastric apparatus including an applier having a distal end and a proximal end. The applier further includes two gastric wall engagement elements opposing one another and in cooperative arrangement for engaging an anterior gastric wall and a posterior gastric wall into abutment and deploying a plurality of tissue fasteners, simultaneously or sequentially. and adapted for applying the tissue fasteners to fix the anterior and posterior gastric walls together for the creation of a septum within a stomach. The septum allows for fluid communication between cavities through a pathway defined by the septum adjacent the antrum.

### CROSS-REFERENCE TO RELATED APPLICATION

This application is a national phase application of PCT Application No. PCT/US2022/032968, internationally filed on Jun. 10, 2022, which claims the benefit of Provisional Application No. 63/213,364, filed Jun. 22, 2021, which are incorporated herein by reference in their entireties for all purposes.

### FIELD

The present disclosure relates generally to apparatuses, systems, and methods for partially compartmentalizing a stomach. More specifically, the disclosure relates to apparatuses, systems, and methods for a non-cutting bariatric coupling device and method to partially compartmentalize a stomach.

### BACKGROUND

There is a need to implement treatments and preventative measures to decrease the prevalence of obesity and the subsequent diseases that obesity can cause. One of the treatments that is established in the field is the sleeve gastrectomy. This procedure involves the removal of about 75% of the stomach in order to restrict the size of the stomach and cause the patient to feel fuller faster. This procedure is typically accomplished with a laparoscopic procedure in which a surgeon staples and dissects a portion of the stomach to form a smaller stomach. This procedure has several known complications that may increase the cost and recovery from the procedure for the patient, such as gastric leaks and bleeding. One such complication is bleeding from the point of dissection of the stomach. Further, the procedure is not reversable. There continues to be a need for a treatment modality that has fewer surgical complications and may be reversible.

### SUMMARY

The present disclosure describes a method and an apparatus configured to partially compartmentalize a stomach. This separates the body cavity of the stomach to be divided into a first and restricted stomach but only partially, resulting in a pathway between the two cavities for fluid communication. The compartmentalization allows for a smaller body cavity portion to be formed that is meant to restrict the amount of food that the patient needs to consume before feeling full, while not removing a large portion of the stomach which could cause complications such as fluid leakage.

According to one example (“Example 1”), an endoscopic gastric apparatus includes an applier having a distal end and a proximal end, the applier including two gastric wall engagement elements opposing one another and in cooperative arrangement for engaging an anterior gastric wall and a posterior gastric wall into abutment and deploying a plurality of tissue fasteners, simultaneously or sequentially, operable for applying the tissue fasteners to fix the anterior and posterior gastric walls together for the creation of a septum within a stomach defining an active stomach portion and a restricted stomach portion, wherein the septum allows for fluid communication between the active stomach portion and the restricted stomach portion through a pathway defined by the septum.

According to a second example (“Example 2”), the endoscopic gastric apparatus of Example 1 includes wherein the applier is secured at the distal end with a support shaped and dimensioned for passage into an abdominal cavity.

According to a third example (“Example 3”), the endoscopic gastric apparatus of Example 1 includes wherein the septum extends from a fundus to adjacent an antrum such that the pathway define by the septum is adjacent the antrum.

According to a fourth example (“Example 4”), the endoscopic gastric apparatus of Example 1 includes wherein each of the plurality of tissue fasteners are deployed sequentially.

According to a fifth example (“Example 5”), the endoscopic gastric apparatus of Example 1 includes wherein each of the plurality of tissue fasteners are deployed simultaneously.

According to a sixth example (“Example 6”), the endoscopic gastric apparatus of Example 1 includes wherein the pathway is sized as to allow for passage of substances from the active stomach portion and the restricted stomach portion.

According to a seventh example (“Example 7”), the endoscopic gastric apparatus of Example 1 includes wherein the plurality of tissue fasteners is at least one of a staple, suture, rivet, adjustable rivet, and opposing magnets.

According to an eighth example (“Example 8”), the endoscopic gastric apparatus of Example 1 includes wherein a number of the plurality of tissue fasteners deployed ranges from approximately 1 to 40.

According to a ninth example (“Example 9”), the endoscopic gastric apparatus of Example 1 includes wherein the apparatus comprises a first arced portion and a second arced portion at a proximal end of each of the two gastric wall engagement elements.

According to a tenth example (“Example 10”), the endoscopic gastric apparatus of Example 9 includes wherein the first and second arced portions are configured to enclose a spacing when the apparatus is in a closed configuration, the spacing operable to form the pathway defined by the septum.

According to an eleventh example (“Example 11”), the endoscopic gastric apparatus of Example 1 includes wherein a first of the two gastric wall engagement elements comprises a cartridge housing the plurality of tissue fasteners.

According to a twelfth example (“Example 12”), the endoscopic gastric apparatus of Example 1 includes wherein a second of the two gastric wall engagement elements comprises of a plurality of tissue fastener receiving elements.

According to a thirteenth example (“Example 13”), the endoscopic gastric apparatus of Example 12 includes wherein the plurality of tissue fasteners comprises staples and the plurality of tissue fastener receiving elements comprises staple pockets.

According to a fourteenth example (“Example 14”), the endoscopic gastric apparatus of Example 1 includes wherein the two gastric wall engagement elements are configured to each have a length that is approximately between 200 mm and 250 mm.

According to a fifteenth example (“Example 15”), the endoscopic gastric apparatus of Example 1 includes wherein the two gastric wall engagement elements are configured to each have a length that are approximately equal.

According to a sixteenth example (“Example 16”), a method for partially compartmentalizing a stomach includes accessing an anterior and a posterior gastric wall between a lesser curvature and a greater curvature, engaging the anterior and posterior gastric walls toward each other in abutment at a location defining an active stomach portion and a restricted stomach portion, and coupling the anterior and posterior gastric wall so as to secure the abutment and forming a septum from the fundus to adjacent the antrum while retaining fluid communication between the active stomach portion and the restricted stomach portion.

According to a seventeenth example (“Example 17”), the method for partially compartmentalizing a stomach of Example 16 further includes wherein the location at which the posterior and anterior gastric are engaged towards each other is from a fundus to adjacent an antrum.

According to an eighteenth example (“Example 18”), the method for partially compartmentalizing a stomach of Example 17, wherein during the coupling step, the septum is formed from the fundus to adjacent the antrum.

According to a nineteenth example (“Example 19”), the method for partially compartmentalizing a stomach of Example 16 includes wherein coupling includes a means for puncturing the anterior and the posterior gastric wall and securing with a tissue fastener.

According to a twentieth example (“Example 20”), the method for partially compartmentalizing a stomach of Example 16 includes wherein coupling includes at least one of stapling, suturing, and riveting.

According to a twenty-first example (“Example 21”), the method for partially compartmentalizing a stomach of Example 16 includes wherein after the coupling of the anterior and the posterior gastric wall, the active stomach portion and the restricted stomach portion remain coupled.

According to a twenty-second example (“Example 22”), the method for partially compartmentalizing a stomach of Example 18 includes wherein the septum formed from the coupling has a length that extends a majority of a length from the fundus to the antrum.

According to a twenty-third example (“Example 23”), the method for partially compartmentalizing a stomach of Example 16 further includes wherein the septum formed from the coupling has a length that extends completely a length from a fundus to an antrum, wherein the septum defines fluid pathways at one or more locations along the septum.

According to a twenty-fourth example (“Example 24”), the method for partially compartmentalizing a stomach of Example 16 includes wherein coupling of the anterior and posterior gastric wall is completed through the use of an apparatus comprising two gastric wall engagement elements opposing one another.

According to a twenty-fifth example (“Example 25”), the method for partially compartmentalizing a stomach of Example 16 includes wherein accessing the anterior and posterior gastric wall further includes positioning a first of the two gastric wall engagement elements adjacent the anterior gastric wall and positioning a second of the two gastric wall engagement elements adjacent the anterior gastric wall.

According to a twenty-sixth example (“Example 26”), the method for partially compartmentalizing a stomach of Example 25 includes wherein engaging the anterior and the posterior gastric walls further includes engaging the first and the second gastric wall engagement elements towards one another.

According to a twenty-seventh example (“Example 27”), the method for partially compartmentalizing a stomach of Example 24 includes wherein the coupling further includes deployment of a plurality of tissue fasteners from one of the two gastric wall engagement elements.

According to a twenty-eighth example (“Example 28”), the method for partially compartmentalizing a stomach of Example 24 includes coupling further includes deployment of a tissue fastener from one of the two gastric wall engagement elements.

According to a twenty-ninth example (“Example 29”), the method for partially compartmentalizing a stomach of Example 28 includes wherein coupling further includes repositioning the two gastric wall engagement elements and deploying another tissue fastener of a plurality of tissue fasteners.

According to a thirtieth example (“Example 30”), the method for partially compartmentalizing a stomach of Example 24 includes removing the apparatus from adjacent the anterior and posterior gastric walls without severing the anterior and posterior gastric walls.

According to a thirty-first example (“Example 31”), a method for treating obesity includes using the endoscopic gastric apparatus of any one of Examples 1 to 15 to engage the anterior and posterior gastric walls towards each other in abutment at a location from a fundus to adjacent an antrum defining an active stomach portion and a restricted stomach portion and coupling the anterior and posterior gastric wall so as to secure the abutment and forming a septum from the fundus to adjacent the antrum while retaining fluid communication between the active stomach portion and the restricted stomach portion.

According to a thirty-second example (“Example 32”), the method for treating obesity of Example 31 includes wherein the septum is formed from the fundus to the antrum.

According to a thirty-third example (“Example 33”), the method for treating obesity of Example 31 includes wherein the fluid communication is retained through a pathway defined by the septum and a stomach wall.

According to a thirty-fourth example (“Example 34”), the method for treating obesity of Example 31 includes wherein the coupling of the anterior and posterior gastric wall includes positioning one of the two gastric wall engagement elements adjacent the anterior gastric wall and the other of the two gastric wall engagement elements adjacent the posterior gastric wall and deploying the one or more plurality of tissue fasteners.

According to a thirty-fifth example (“Example 35”), a method for treating diabetes includes using the endoscopic gastric apparatus of any one of Examples 1 to 15 to engage the anterior and posterior gastric walls towards each other in abutment at a location from a fundus to adjacent an antrum defining an active stomach portion and a restricted stomach portion and coupling the anterior and posterior gastric wall so as to secure the abutment and forming a septum from the fundus to adjacent the antrum while retaining fluid communication between the active stomach portion and the restricted stomach portion.

According to a thirty-sixth example (“Example 36”), the method for treating diabetes of Example 35 includes wherein coupling the anterior and posterior gastric wall includes positioning one of the two gastric wall engagement elements adjacent the anterior gastric wall and the other of the two gastric wall engagement elements adjacent the posterior gastric wall and deploying the one or the plurality of tissue fasteners.

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a perspective view of an upper gastrointestinal system of a patient, in accordance with an embodiment; and

FIG. 2A is an enlarged side view of a portion of the upper gastrointestinal system of the patient shown in FIG. 1; and

FIG. 2B is a rear view of the enlarged side view of a portion of the upper gastrointestinal system as shown in FIG. 2A; and

FIG. 2C is an enlarged side view of a portion of the upper gastrointestinal system of the patient shown in FIG. 1; and

FIG. 2D is an enlarged side view of a portion of the upper gastrointestinal system of the patient shown in FIG. 1; and

FIG. 2E is a side cross-sectional view taken along line 2E-2E of the partially-compartmentalized stomach of FIG. 2A, in accordance with an embodiment; and

FIG. 2F is a side cross-sectional view taken along line 2F-2F of the partially-compartmentalized stomach of FIG. 2A, in accordance with an embodiment;

FIG. 3 is a side view of an endoscopic gastric apparatus, in accordance with an embodiment; and

FIG. 4 is an enlarged side view of a portion of the endoscopic apparatus of FIG. 3, in accordance with an embodiment; and

FIG. 5A is a side view of an endoscopic gastric apparatus, in accordance with an embodiment; and

FIG. 5B is a side view of a portion of the endoscopic gastric apparatus of FIG. 5A, in accordance with an embodiment; and

FIG. 6 is a side view of an endoscopic gastric apparatus, in accordance with an embodiment; and

FIG. 7A illustrates various tissue fasteners before being deployed, in accordance with an embodiment; and

FIG. 7B illustrates various tissue fasteners after being deployed, in accordance with an embodiment; and

FIG. 7C illustrates a top view of the various tissue fasteners of FIG. 7A, in accordance with an embodiment; and

FIG. 8 is a flow chart illustrating a method for partially compartmentalizing a stomach, in accordance with an embodiment.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatuses configured to perform the intended functions, such as, but not limited to, limiting fluid communication between portions of the stomach. Such limiting of fluid communication may be beneficial as a means for treating obesity, and secondarily may be a means for treating or preventing diabetes. It should also be noted that the accompanying drawing figures referred to herein are not necessarily drawn to scale, but may be exaggerated to illustrate various aspects of the present disclosure, and in that regard, the drawing figures should not be construed as limiting.

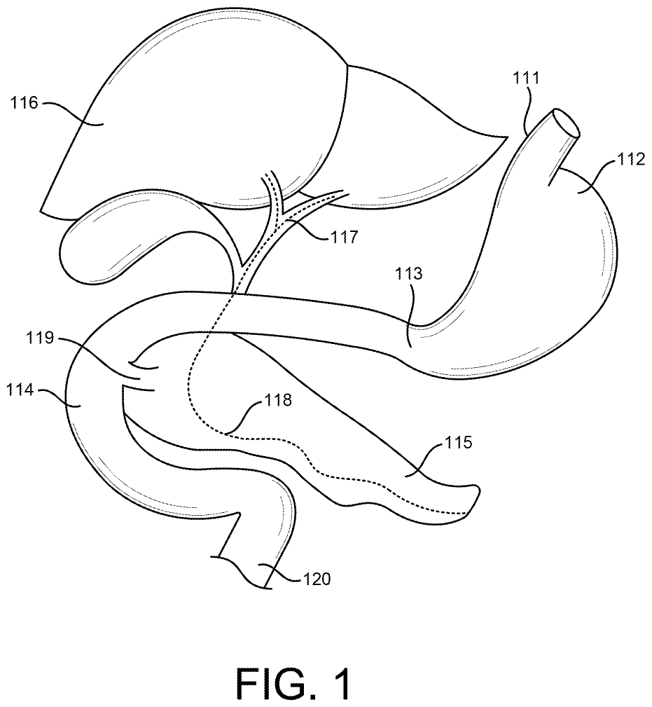

FIG. 1 is a perspective view of an upper gastrointestinal system. After swallowing, food passes rapidly through the esophagus 111 into the stomach 112. There, it is digested for a period of time and undergoes the process of dilution to an iso-osmotic concentration by grinding and mixing with gastric juices. The stomach 112 relaxes to accommodate the volume of ingested food. Fluid restriction within the stomach 112 may facilitate weight loss. In certain instances, as the stomach 112 gets filled with food the sensation of fullness or satiety is generated by stretch receptors in the gastric wall and the person stops eating. The iso-osmotic food, known as chyme, then passes through the pylorus 113 into the duodenum 114. Passage of chyme into the duodenum 114 results in the release of enzyme rich pancreatic secretions from the pancreas 115 and bile salt rich biliary secretions from the liver 116. The biliary secretions travel through the common bile duct 117 where they combine with the pancreatic secretions arriving through the pancreatic duct 118 and the two ducts combine to form the ampulla of vater 119. The ampulla of vater 119 serves as the entry point for the secretions to be deposited into the duodenum 114. In the jejunum 120, the mixing of pancreatic and biliary secretions with the chyme results in the digestion of proteins, fats, and carbohydrates, which are then absorbed into the blood stream.

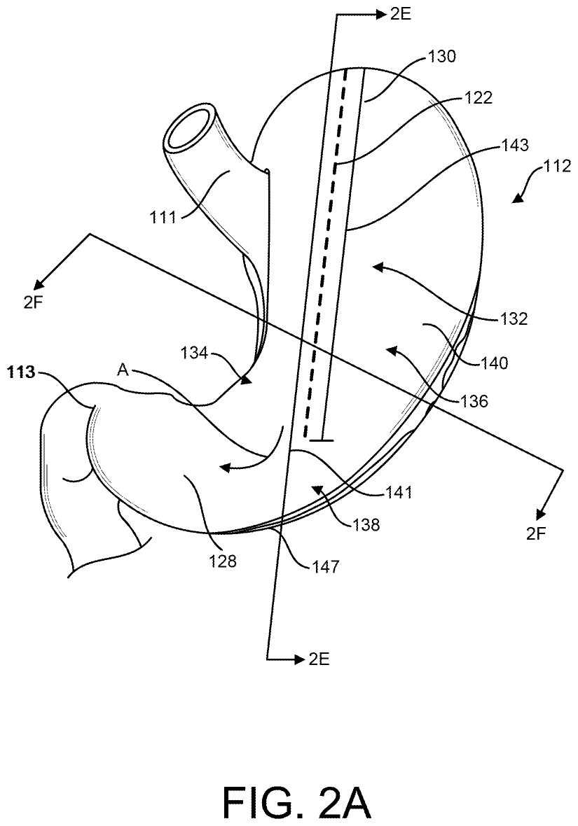

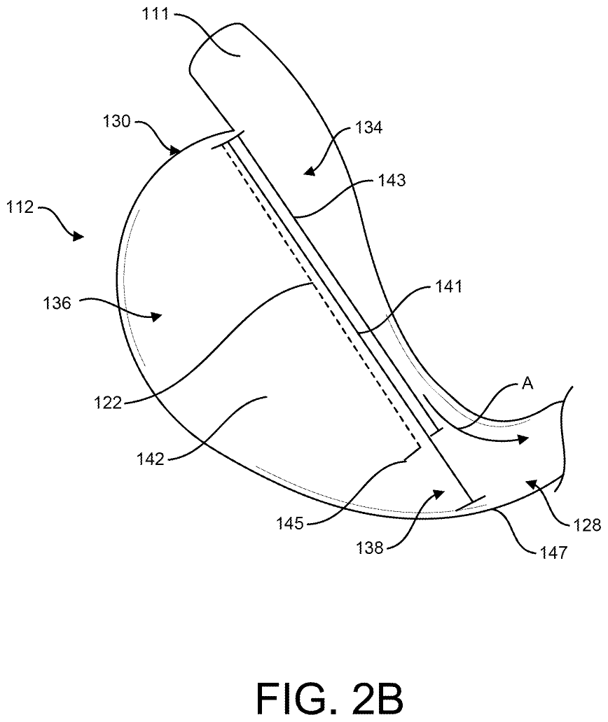

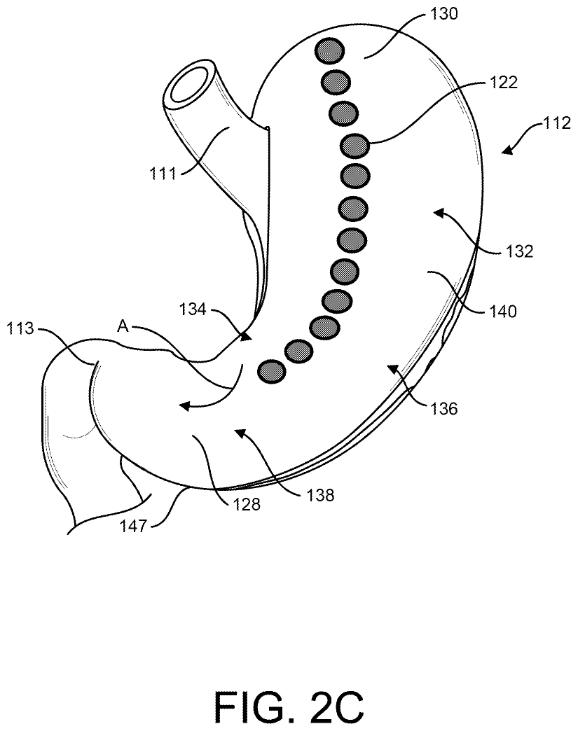

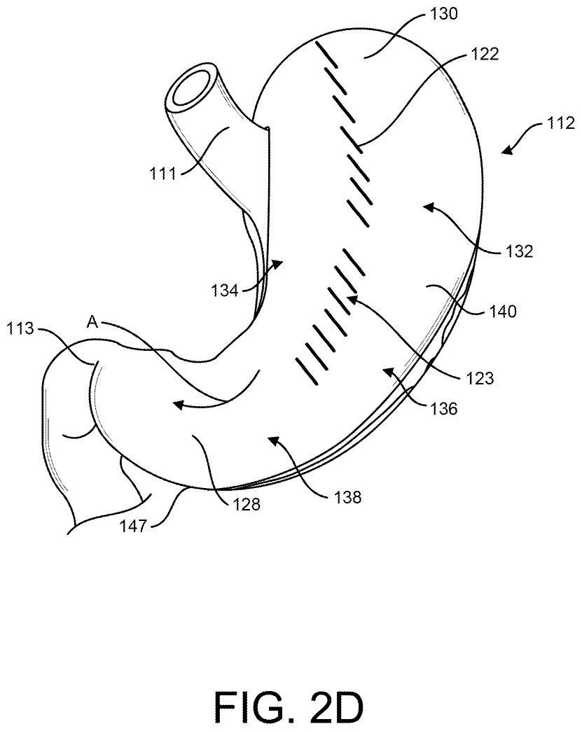

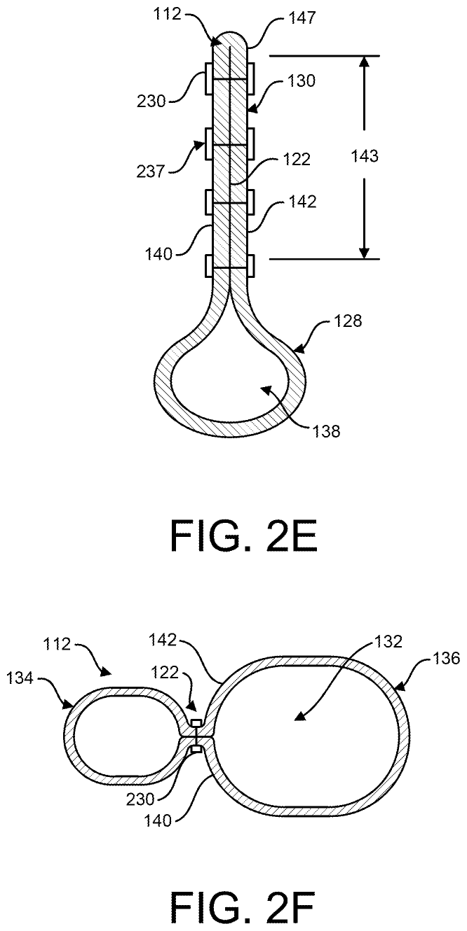

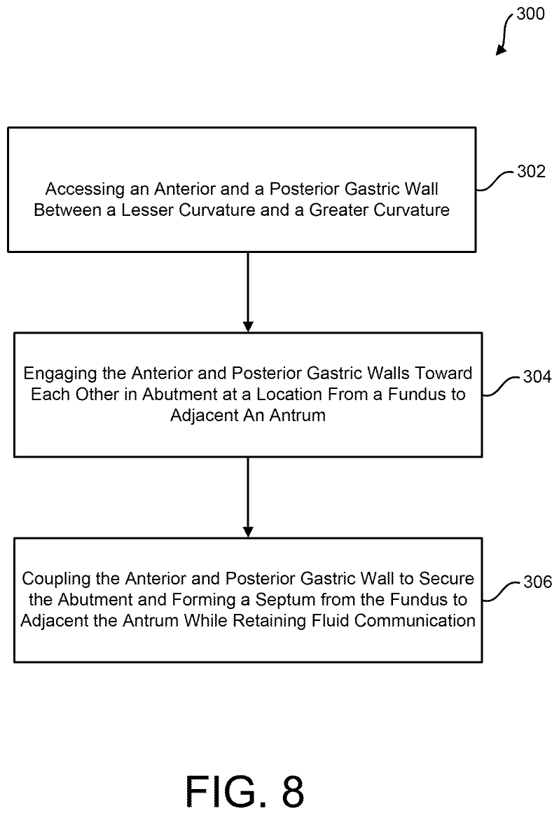

FIG. 2A is a side view of the stomach illustrated in FIG. 1, after being partially compartmentalized by the surgically-created septum 122, for which methods will be described with reference to FIG. 8. The partial compartmentalization of the stomach 112 simulates the desired result of the Magenstrasse Mill procedure, but it does not require the surgical separation of medial and lateral portions of the stomach 112, as is required in the Magenstrasse Mill procedure. This may contribute to reduced complications and reversibility of the present procedure, as described previously. The stomach 112 includes a fundus 130 defined by the uppermost portion of the stomach 112, and an antrum 128 defined by the lowermost portion of the stomach 112. The body cavity 132 of the stomach 112 is divided by the surgically-created septum 122 which defines an active stomach portion 134 and a restricted stomach portion 136 of the stomach 112. The stomach 112 also has an anterior gastric wall 140 and a posterior gastric wall (not shown). The surgically-created septum 122 is formed by the urging engagement of a portion of the anterior gastric wall 140 and a posterior gastric wall 142 (FIG. 2E) generally along a line. As provided below, the urging engagement of a portion of the anterior gastric wall 140 and a posterior gastric wall 142 (FIG. 2E) may be facilitated by the placement of a line of staples, suture, rivets, among other fastening means, that are operable to bring the tissue into opposition and engagement.

Various aspects of the present disclosure are directed toward forming the septum 122 that partially compartmentalizes the stomach 112 to define an active stomach portion 134 and a restricted stomach portion 136 which remain in fluid communication with each other. In accordance with an embodiment, the septum 122 is a surgically-created feature that creates a partial wall from the fundus 130 toward the antrum 128. In another embodiment, the septum 122 is a surgically-created feature that creates a wall between the fundus 130 and the antrum 128 defining an active stomach portion 134 and a restricted stomach portion 136 but defines intermittent fluid passages 123 there between, as will be described with reference to FIG. 2D. The active stomach portion 134 may be smaller than the restricted stomach portion 136. In other instances, the active stomach portion 134 may be approximately equal in size to the restricted stomach portion 136.

In accordance with these embodiments, the fluid communication between the active stomach portion 134 and a restricted stomach portion 136 is provided for a particular purpose. By way of example, but not limited thereto, fluid communication allows for the drainage of gastric enzymes from the restricted stomach portion 136 to the active stomach portion 134. By way of another example, but not limited thereto, fluid communication allows for an equalization of gastric pressure between the active stomach portion 134 and the restricted stomach portion 136 with a corresponding reduction of wall pressure at the septum 122. An equalization of gastric pressure may also reduce gastroesophageal reflux disease (GERD). Further and without limitation, fluid communication between the active stomach portion 134 and the restricted stomach portion 136 by way of openings or gaps in an intermittent septum allows for the free flow of blood in the tissues of the restricted stomach portion 136 which prevents necrosis. This is important to not only retain viable stomach functionality, but also may allow for the procedure to be reversable by deconstructing the septum 122. Deconstructing the septum 122 may be facilitated by removing or disengaging the fastening means forming the septum 122, such as, but not limited to removing the staples, cutting the suture, removal of rivets, and the disintegration of the fastening means if it were to be made from a biodegradable or bioabsorbable material.

As previously described with reference to FIG. 1, food passes through the esophagus 111 into the stomach 112. In this instance, the food will pass into the active stomach portion 134 and thus the amount of food that may enter the stomach is physically restricted in comparison to prior to compartmentalizing. Besides the physical limitation of the amount of food that may be consumed, the patient may also feel full with less food as experienced before. In accordance with an embodiment, the septum 122 has a length 143 that spans a majority of a length 141 of the stomach 112 such that a pathway 138 is defined between the active stomach portion 134 and the restricted stomach portion 136 operable to maintain fluid communication. This may be beneficial for the passage of gastric enzymes or other substances formed in the restricted stomach portion 136, such as, but not limited to, pepsin, gastric lipase, hydrochloric acid and mucin, to pass to the active stomach portion 134. This fluid transfer may reduce necrosis of the tissue of the restricted stomach portion 136, as a result of digestive enzymes remaining in the restricted stomach portion 136 for extended periods of time. Some of the substances, such as hydrochloric acid, are highly acidic and may cause damage to the restricted stomach portion 136 if not able to exit the stomach 112.

The septum 122 is configured such that it guides and restricts food to only pass through the active stomach portion 134 with the pathway 138 sized such that it is unlikely for food to pass through the pathway 138 from the active stomach portion 134 to the restricted stomach portion 136. Rather, the food passes along the direction of an arrow A illustrated in FIG. 2A, which illustrates movement through the active stomach portion 134 and generally away from the restricted stomach portion 136. It is appreciated that the size of the pathway 138 is predetermined to be small enough to restrict food passage into the restrictive stomach portion but large enough to allow for fluid to pass between the restricted stomach portion 136 and the active stomach portion 134. In various instances, the septum 122 is generally linear. In other instances, the septum 122 may be generally curved for reasons, such as, but not limited to, mimicking the anatomy of the patient. The shape of the septum 122 may be dependent on at least the shape of the endoscopic gastric apparatus 200, as will be described further herein.

FIG. 2B is an additional view of the expanded side view of the stomach 112. The stomach 112 comprises the septum 122 configured to compartmentalize the stomach 112 into the active stomach portion 134 and the restricted stomach portion 136. The pathway 138 is defined as the space between an end 145 of the septum 122 and the adjacent a stomach wall 147 of the antrum 128. The septum 122 spans the length 143 along the stomach 112 beginning at the fundus 130 to adjacent the stomach wall 147 of the antrum 128. The stomach 112 further comprises the anterior gastric wall 140 (FIG. 2A) and the posterior gastric wall 142. Similar to in FIG. 2A, the arrow A illustrates the direction in which food may now pass through the active stomach portion 134 as a result of the pathway 138 being sized to reduce to the ability of food to pass from the active stomach portion 134 to the restricted stomach portion 136.

FIG. 2C is a side view of the stomach illustrated in FIG. 1, after being partially compartmentalized by the surgically-created septum 122. Similar to the stomach 112 displayed in FIG. 2A, the septum 122 compartmentalizes the body cavity 132 of the stomach 112 into the restricted stomach portion 136 and the active stomach portion 134. In this embodiment, the septum 122 still spans generally from the fundus 130 to adjacent the antrum 128. The septum 122 is illustrated as generally curved. The pathway 138 is retained between the antrum 128 and the septum 122.

FIG. 2D is a side view of the stomach illustrated in FIG. 1, after being partially compartmentalized by the surgically-created septum 122. Similar to the stomach 112 illustrated in FIGS. 2B and 2C, the septum 122 compartmentalizes the body cavity 132 to form the restricted stomach portion 136 and the active stomach portion 134 of the stomach 112. In this embodiment, the stomach 112 is partially compartmentalized by the surgically-created septum 122 having a plurality of intermittent fluid passages 123 for fluid communication along the septum 122. This may allow small amounts of fluid to pass between the septum 122 from the active stomach portion 134 to the restricted stomach portion 136 and from the restricted stomach portion 136 to the active stomach portion 134.

FIG. 2E is a side cross-sectional view of the stomach 112 of FIGS. 2A and 2B taken along line 2E-2E of FIG. 2A. The stomach 112 comprises the surgically-created septum 122 formed by the urging engagement of a portion of the anterior gastric wall 140 and the posterior gastric wall 142. As previously disclosed, the urging of the anterior and posterior gastric walls 140, 142 may be facilitated by the placement of one or a plurality of tissue fasteners 230. The plurality of tissue fasteners 230 are illustrated generally as rivets 237. The positioning of the plurality of tissue fasteners 230 is configured for retaining the pathway 138 available for fluid communication between the active and restricted stomach portions 134, 136 (FIG. 2A). As illustrated in this embodiment, the septum 122 is defined by the length 143 such that below the septum 122, the pathway 138 remains. While the urging engagement of the anterior gastric wall 140 and the posterior gastric wall 142 must be with sufficient compression to create a seal, it should be noted that too much compression may cause a risk of reduced blood flow to the tissue. It is thus desired that the compression used when urging the anterior and posterior gastric walls 140, 142 together is large enough to seal the active and restricted stomach portions 134, 136 (FIG. 2B) but still maintains the viability of the tissue of the anterior and posterior gastric walls 140, 142. This allows the tissue to retain its natural function if the partial compartmentalization is reversed.

FIG. 2F is a side cross-sectional view of the stomach 112 of FIGS. 2A and 2B, taken along a line 2F-2F of FIG. 2A. FIG. 2F illustrates a lateral cross section of the stomach. The body cavity 132 of the stomach 112 comprises the active stomach portion 134 and the restricted stomach portion 136, the active and restricted stomach portions 134, 136 separated by the septum 122 formed from at least one of a plurality of tissue fasteners 230. The septum 122 is formed from urging engagement of the anterior gastric wall 140 and the posterior gastric wall 142 through the use of the at least one of the plurality of tissue fasteners 230. Additionally, as illustrated, the active stomach portion 134 is substantially smaller than the restricted stomach portion 136 due to the positioning of the septum 122.

Further, in various embodiments, it may be desired for the partial compartmentalizing to be formed from the implementation of two lateral layers of compression within the septum 122. The compression used when urging the anterior and posterior gastric walls 140, 142 into engagement may be lighter in this instant as a result of having two layers of sealing, such that tissue viability is retained.

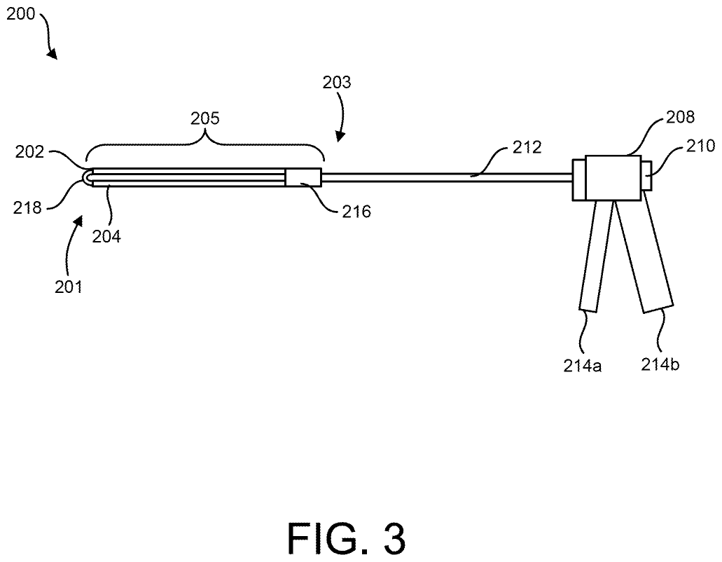

FIG. 3 is a side perspective view of an exemplary embodiment of an endoscopic gastric apparatus 200. The endoscopic gastric apparatus 200 is configured for providing a fastening means so as to bring the anterior gastric wall 140 into urging engagement with the posterior gastric wall 142 and fixed there to, partially compartmentalizing the stomach 112 by forming a septum 122, as illustrated in FIGS. 2A and 2B, and thus will be additionally described with reference to FIGS. 2A and 2B.

The endoscopic gastric apparatus 200 includes an applier 205 with a distal end 201 and a proximal end 203, wherein the applier 205 is secured at the distal end 201 with a support element 218. In certain instances, the support element 218 is a flexible lock attached to distal end 201 through a hinge, such that the support element 218 can reversibly lock and unlock from the endoscopic gastric apparatus 200. The support element 218 is shaped and dimensioned for passage into an abdominal cavity (not shown) before accessing the stomach 112. The endoscopic gastric apparatus 200 may include a handle assembly 208 that includes a first grasping component 214a and a second grasping component 214b for operation of the endoscopic gastric apparatus 200. The endoscopic gastric apparatus 200 further includes an actuator 210 for deploying a coupling mechanism of the applier 205.

The endoscopic gastric apparatus 200 further includes a first gastric wall engagement element 202 and a second gastric wall engagement element 204. The first gastric wall engagement element 202 and second gastric wall engagement element 204 are configured to oppose one another and are positioned in cooperative arrangement for engaging the anterior gastric wall 140 and the posterior gastric wall 142 of the stomach 112 (FIG. 2A) into abutment. The first gastric wall engagement element 202 and the second gastric wall engagement element 204 cooperate to apply the one or more tissue fasteners (not shown) to retain the coupling of the anterior and posterior gastric walls 140, 142, respectively, of the stomach 112 and to urgingly engage the anterior and posterior gastric walls 140, 142 together to form the septum 122. The application of the one or more tissue fasteners (not shown) may be accomplished mechanically or electrically through the use of the actuator 210 of the endoscopic gastric apparatus 200.

Further, the endoscopic gastric apparatus 200 includes a connecting portion 212 to connect the proximal end 203 of the applier 205 to the handle assembly 208 of the endoscopic gastric apparatus 200.

The system shown in FIG. 3 is provided as an example of the various features of the endoscopic gastric apparatus 200 and, although the combination of those illustrated features is clearly within the scope of invention, that example and its illustration is not meant to suggest the inventive concepts provided herein are limited from fewer features, additional features, or alternative features to one or more of those features shown in FIG. 3. For example, the handle assembly 208 of the endoscopic gastric apparatus 200 shown in FIG. 3, may include other grasping mechanisms and configurations known in the art that aid in the operation of the endoscopic gastric apparatus 200. Additionally, the support element 218 may be any element that may be configured for the retention of the first gastric wall engagement element 202 and second gastric wall engagement element 204 at the distal end 201 of the endoscopic gastric apparatus 200 and shaped to pass through the abdominal cavity (not shown).

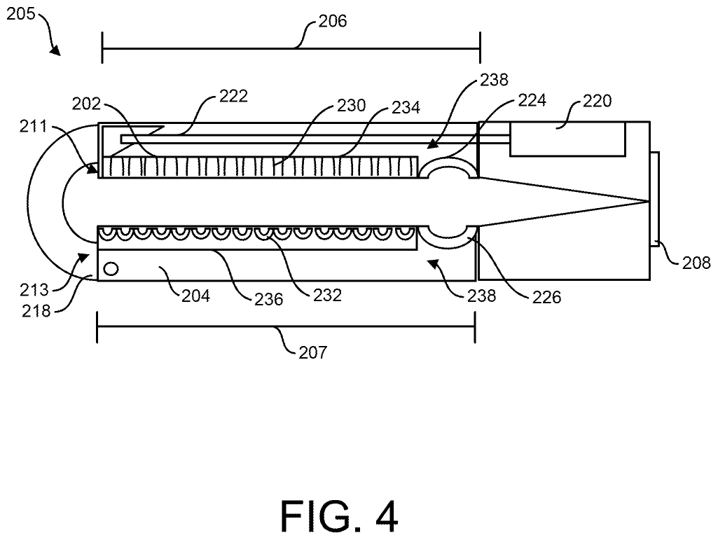

FIG. 4 is an expanded side view of the applier 205 of the endoscopic gastric apparatus 200 illustrated in accordance with the instance of FIG. 3. FIG. 4 illustrates the first gastric wall engagement element 202 positioned opposite the second gastric wall engagement element 204. In certain instances, the first gastric wall engagement element 202 may include a first cartridge 234 containing the one or more tissue fasteners 230. In certain instances, the one or more tissue fasteners 230 may include one of a staple, suture, rivet, adjustable rivet, multi-headed rivet, T-Tag, and a magnet. In various instances, the one or more tissue fasteners 230 may include a plurality of tissue fasteners 230. In other instances, the plurality of tissue fasteners 230 may be any structure that can puncture the anterior gastric wall 140 and the posterior gastric wall 142 and couple them together. In the illustrated instance of FIG. 3 and FIG. 4, the first gastric wall engagement element 202 and the second gastric wall engagement element 204 are relatively linear, and the one or the plurality of tissue fasteners 230 may be deployed relatively linearly to thus form a linear septum 122. In other instances, the first gastric wall engagement element 202 and the second gastric wall engagement element 204 are relatively curved. Thus, the one or the plurality of tissue fasteners 230 may be deployed in a relatively curved arrangement and form a curved septum 122 after being deployed, as illustrated in FIG. 2C.

In some instances, the one or the plurality of tissue fasteners 230 are chosen in order to create the septum 122 having various openings along the septum 122. These may be formed by predetermined spaces between each of the plurality of tissue fasteners 230 when deployed. There can be points of leakage along the septum 122 between the active stomach portion 134 and the restricted stomach portion 136. This may be beneficial in allowing for the passage of various gastric enzymes and fluids, as previously described with reference to the pathway 138. In these instances, the size of the pathway 138 may be reduced, and the size of the septum 122 increased, while still allowing for fluid passage through the septum 122.

In certain instances, the second gastric wall engagement element 204 includes a second cartridge 236 composed of one or a plurality of tissue fastener receiving elements 232. In certain instances, the one or the plurality of tissue fasteners 230 include staples and the one or the plurality of tissue fastener receiving elements 232 include staple pockets. The number of the plurality of tissue fasteners 230 may be chosen based on the number of fasteners required to span a length of 180 cm to 200 cm, which is approximately the length 143 of the septum 122. This number of plurality of tissue fasteners 230 required may range based on the type and size of the tissue fastener used. In some instances, the number of the plurality of tissue fasteners 230 deployed may range from 1 to 40. In other instances, the plurality of tissue fasteners 230 used are staples and the number of staples required may be at least 150 staples. The one or the plurality of tissue fasteners 230 are deployed to create the septum 122 and provide a seal between the active stomach portion 134 and the restricted stomach portion 136 of the stomach 112. While partially compartmentalizing the stomach 112, the one or the plurality of tissue fasteners 230 also limit substances from passing between the active stomach portion 134 and the restricted stomach portion 136. Thus, the number of the tissue fasteners 230 may additionally be chosen to ensure a sufficient seal and reduction of food passage between the active stomach portion 134 and the restricted stomach portion 136. The one or the plurality of tissue fasteners 230 are capable of being removed if desired, allowing the partial compartmentalizing of the stomach 112 to be reversible. Further, the tissue fasteners 230 may be absorbable by the tissue of the patient such that the compartmentalizing of the stomach 112 is reversible without the requirement of further surgical intervention.

The plurality of tissue fasteners 230 may be deployed simultaneously or sequentially. When applied sequentially, the plurality of tissue fasteners 230 deployed may be one tissue fastener at a time. In other instances, when applied sequentially, the plurality of tissue fasteners 230 deployed at a time can be at least two or more at a time. Further, in some instances, a programmed amount of the plurality of tissue fasteners 230 can be chosen to be deployed. Further, the specific tissue fasteners within the plurality of tissue fasteners 230 to be deployed can be chosen in order to customize the length and/or positioning of the septum 122. For example, every other of the plurality of tissue fasteners 230 may be deployed when actuated.

At a proximal end 238 of the first gastric wall engagement element 202 and the second gastric wall engagement element 204, the endoscopic gastric apparatus 200 includes a first arced portion 224 and a second arced portion 226 positioned opposite one another. The first arced portion 224 and the second arced portion 226 are configured such that when the endoscopic gastric apparatus 200 is in a closed configuration, there is a spacing enclosed by the first arced portion 224 and the second arced portion 226. The first arced portion 224 and the second arced portion 226 may not engage the tissue of the anterior and posterior gastric walls 140, 142 and thus can create the pathway 138 positioned below the septum 122 after the one or the plurality of tissue fasteners 230 are deployed.

The first gastric wall engagement element 202 and the first arced portion 224 form a first extendable member 211. The second gastric wall engagement element 204 and the second arced portion 26 form a second extendable member 213. The first extendable member 211 has a first length 206 and the second extendable member 213 has a second length 207. In instances, the values of the first length 206 and the second length 207 range from approximately 200 mm to 250 mm. In certain instances, the first length 206 is approximately equal to the second length 207. In this way, for a typical stomach, the first extendable member 211 and the second extendable member 213 are able to extend across the length 141 of the stomach 112.

The endoscopic gastric apparatus 200 may also include a drive motor 220 and a drive screw 222 that are configured for powering the endoscopic gastric apparatus 200 and drive the deployment of the plurality of tissue fasteners 230 through the anterior and posterior gastric walls 140, 142. In instances, the endoscopic gastric apparatus 200 is the manually driven for deployment of the one or the plurality of tissue fasteners 230.

The system shown in FIG. 4 is provided as an example of the various features of the endoscopic gastric apparatus 200 and, although the combination of those illustrated features is clearly within the scope of invention, that example and its illustration is not meant to suggest the inventive concepts provided herein are limited from fewer features, additional features, or alternative features to one or more of those features shown in FIG. 4. For example, in various instances, the sizing of the first gastric wall engagement element 202 and the second gastric wall engagement element 204, as well as the first arced portion 224 and the second arced portion 226, may be varied.

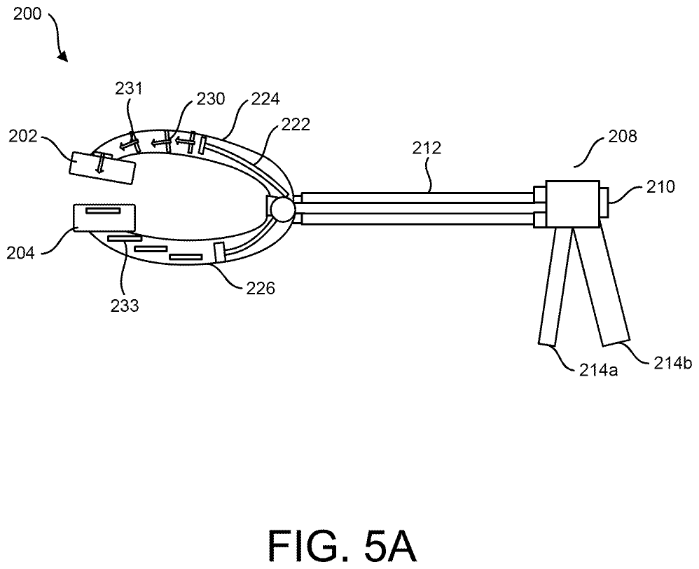

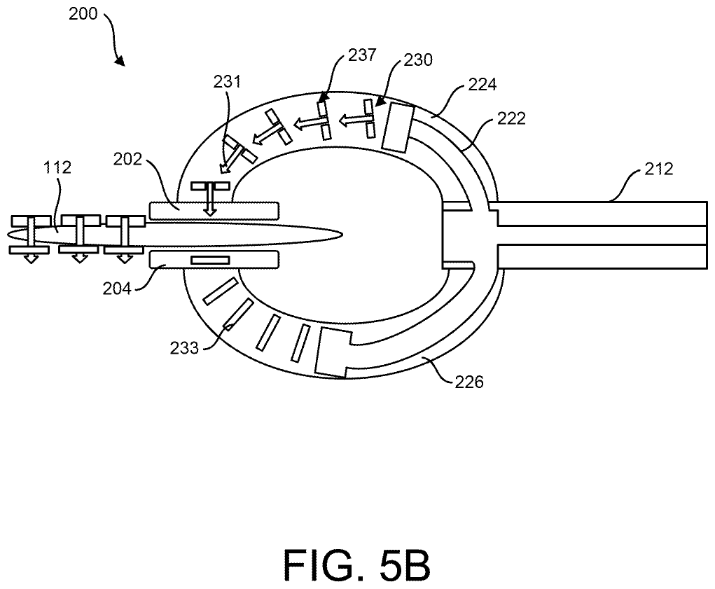

FIG. 5A is an alternative instance of the endoscopic gastric apparatus 200 in accordance with the present disclosure. The endoscopic gastric apparatus 200 includes the handle assembly 208 that includes the first grasping component 214a and the second grasping component 214b for operation of the endoscopic gastric apparatus 200. The endoscopic gastric apparatus 200 further includes the actuator 210 for deploying the plurality of tissue fasteners 230. The endoscopic gastric apparatus 200 further includes the connecting portion 212 including the drive motor (not shown) and the drive screw 222 for driving the deployment of the one or the plurality of tissue fasteners 230 through the anterior and posterior gastric walls 140, 142 (FIG. 2A). The endoscopic gastric apparatus 200 further includes the first gastric wall engagement element 202 and the second gastric wall engagement element 204 positioned opposite the first gastric wall engagement element 202. The endoscopic gastric apparatus 200 additionally includes a first arced portion 224 and a second arced portion 226. The first arced portion 224 is positioned adjacent the first gastric wall engagement element 202 and the second arced portion 226 is positioned adjacent the second gastric wall engagement element 204.

In instances, the plurality of tissue fasteners 230 include a plurality of rivets 237. In these instances, the first arced portion 224 includes a plurality of anterior rivet bodies 231 and the second arced portion 226 includes a plurality of posterior rivet bases 233. Each of the plurality of anterior rivet bodies 231 is deployed from the first gastric wall engagement element 202 and each of the plurality of posterior rivet bases 233 is deployed from the second gastric wall engagement element 204. Each of the plurality of tissue fasteners 230 is deployed sequentially such that one tissue fastener is deployed at a time. The endoscopic gastric apparatus 200 may be repositioned after each deployment of each of the plurality of tissue fasteners 230 in order to create the septum 122 (FIG. 2A). This deployment and repositioning may be repeated until the septum 122 is formed and the tissue remaining between the first arced portion 224 and second arced portion 226 is not coupled. Thus, when the endoscopic gastric apparatus 200 is removed, the tissue that remained uncoupled forms the pathway 138 (FIG. 2A) between the septum 122 and adjacent the antrum 128 (FIG. 2A). Similar to the instance described with reference to the endoscopic gastric apparatus 200 of FIG. 3 and FIG. 4, the septum 122 may be linear or curved, dependent on the manner in which each of the plurality of tissue fasteners 230 is deployed.

Similar to the one or the plurality of tissue fasteners 230 described with reference to the endoscopic gastric apparatus 200 of FIGS. 3 and 4, the number of plurality of tissue fasteners 230 deployed may be chosen such that a sufficient seal created between the anterior and posterior gastric wall 140, 142 has a length of approximately 180 cm to 200 cm. In this way, the number of rivets used may depend on the size of the rivets and the spacing desired between each of the rivets once deployed.

The system shown in FIG. 5A is provided as an example of the various features of the endoscopic gastric apparatus 200 and, although the combination of those illustrated features is clearly within the scope of invention, that example and its illustration is not meant to suggest the inventive concepts provided herein are limited from fewer features, additional features, or alternative features to one or more of those features shown in FIG. 4. For example, in various instances, the sizing of the first gastric wall engagement element 202 and the second gastric wall engagement element 204, as well as the first arced portion 224 and the second arced portion 226, may be varied. Additionally, the one or the plurality of tissue fasteners 230 may include an alternate type of tissue fastener than the plurality of rivets 237 illustrated.

FIG. 5B is an additional side view of the endoscopic gastric apparatus 200 of FIG. 5A after deploying a plurality of tissue fasteners 230 into the stomach 112. As illustrated, the plurality of tissue fasteners 230 in this embodiment comprise the plurality of rivets 237 having a plurality of anterior rivet bodies 231 and a plurality of posterior rivet bases 233, such that the anterior rivet bodies 231 are deployed from the first gastric wall engagement element 202 and the posterior rivet bases 233 are deployed from the second gastric wall engagement element 204. When deployed, the anterior rivet bodies 231 are positioned adjacent the anterior gastric wall 140 (FIG. 2A) and the posterior rivet bases 233 are positioned adjacent the posterior gastric wall 142 (FIG. 2B). The anterior rivet bodies 231 extend from adjacent the anterior gastric wall and extend through and out of the posterior gastric wall 142 of the stomach 112. Additionally, each of the plurality of tissue fasteners 230 in this embodiment are deployed sequentially, such as one at a time, as the endoscopic gastric apparatus 200 is moved along the length 141 (FIG. 2B) of the stomach 112 to create the septum 122 (FIG. 2A).

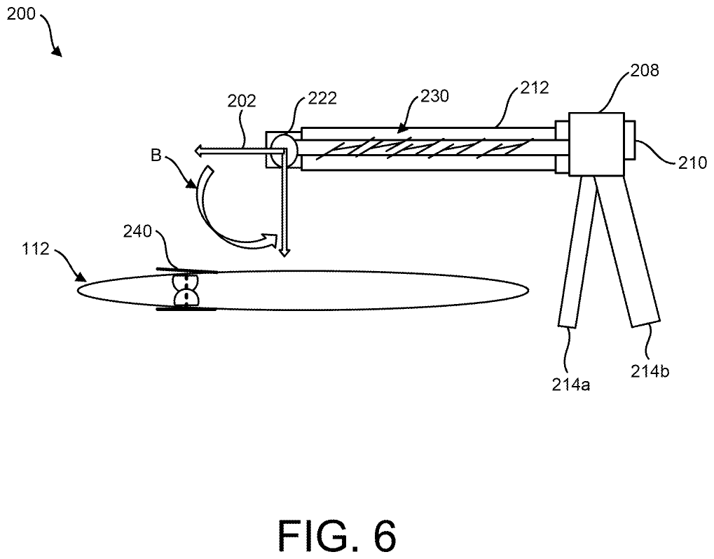

FIG. 6 illustrates an alternative embodiment of the endoscopic gastric apparatus 200. The endoscopic gastric apparatus 200 includes the handle assembly 208 that includes the first grasping component 214a and the second grasping component 214b for operation of the endoscopic gastric apparatus 200. The endoscopic gastric apparatus 200 further includes the actuator 210 for deploying the plurality of tissue fasteners 230. The endoscopic gastric apparatus 200 further includes the connecting portion 212 including the drive motor (not shown) and the drive screw 222 for driving the deployment of the one or the plurality of tissue fasteners 230 through the anterior and posterior gastric walls 140, 142 (FIG. 2A). In this embodiment, the plurality of tissue fasteners 230 is comprised of a plurality of T-Tags 240. Further, in this embodiment, the endoscopic gastric apparatus 200 comprises the first gastric wall engagement element 202 for deploying the plurality of T-Tags 240. The first gastric wall engagement element 202 may begin positioned within the device prior to deployment of the at least one of the plurality of the T-Tags 240. The first gastric wall engagement element 202 may be deployed and rotate in the direction of the arrow B such that the first gastric wall engagement element is positioned generally vertical to deploy at least one of the plurality of T-tags 240. FIG. 6 illustrates the one of the plurality of T-tags 240 positioned through the stomach 112 such that it may form the septum 122 (FIG. 2D).

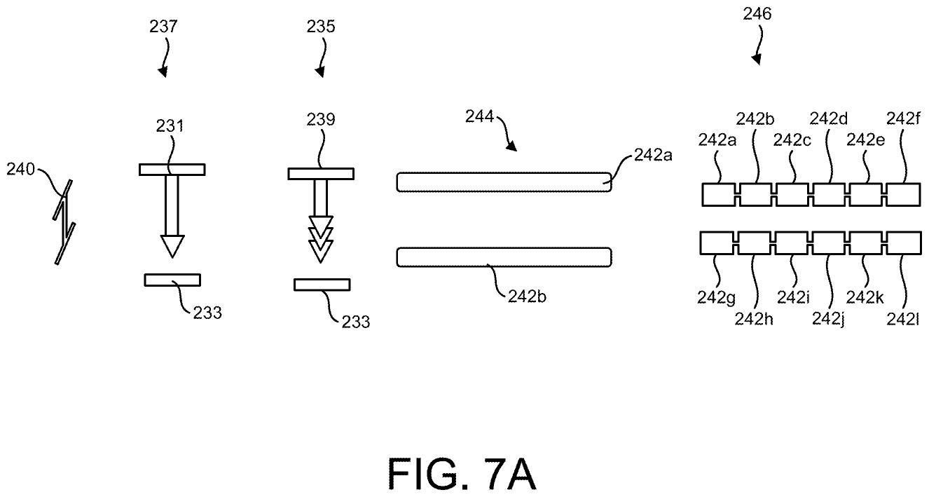

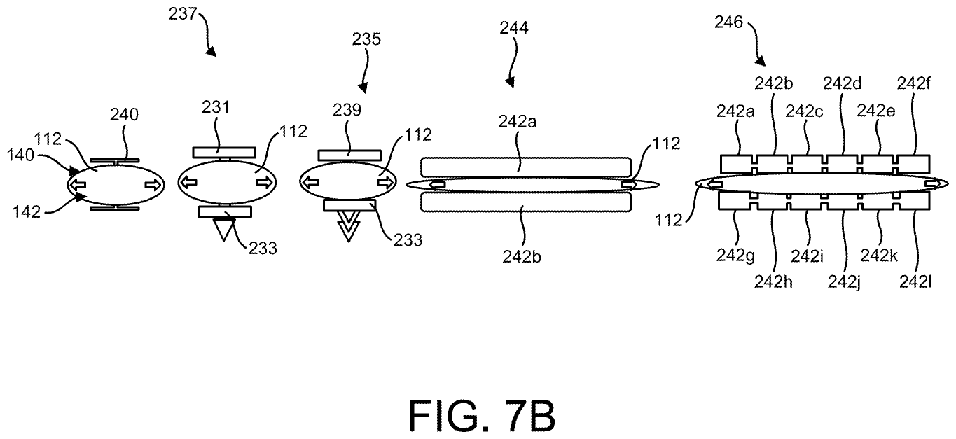

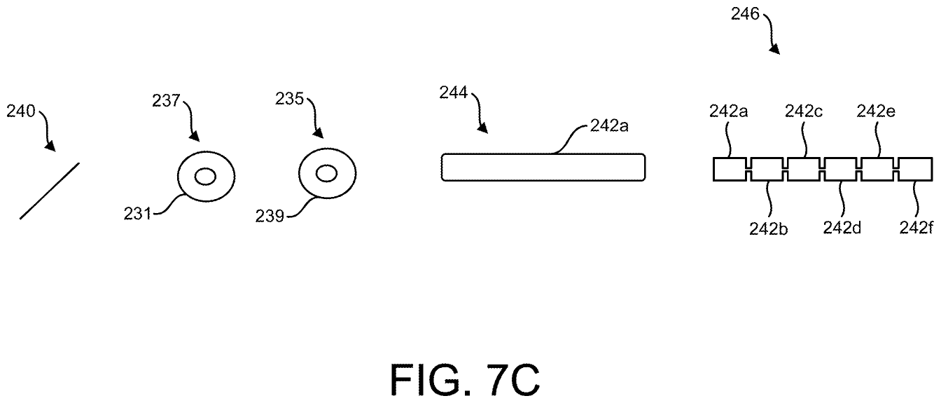

FIG. 7A illustrates various alternative embodiments of a tissue fastener 230 that may be used in accordance with the present disclosure. In this embodiment, the tissue fasteners 230 are illustrated in a pre-deployed state. For example, the one or more tissue fasteners 230 may include the T-tag 240 such that before and during deployment, the T-tag 240 is relatively vertical extending in shape. As described with reference to the embodiment of FIG. 5A, the one or the plurality of tissue fasteners 230 may also comprise a rivet 237 composed of the anterior rivet body 231 and the posterior rivet base 233. Further, the one or the plurality of tissue fasteners 230 may comprise an adjustable rivet 235, such as a multi-headed rivet 235, which comprises a multi-headed anterior rivet body 239 and the posterior rivet base 233. In some instances, the one or the plurality of tissue fasteners 230 may include a magnet 244. The magnet 244 may include at least a first magnet plate 242a and a second magnet plate 242b that are deployed on either side of the stomach 112 (FIG. 7B). Additionally, the one or the plurality of tissue fasteners 230 may include a magnet 246 including three or more magnet plates 242a-l. In various embodiments, such as the illustrated embodiment of FIG. 7A, magnet plates 242a-f may be connected with one another and magnet plates 242g-l may be connected with one another. In this way, the magnet plates 242a-f may be positioned adjacent the anterior gastric wall 140 (FIG. 7B) and the magnet plates 242g-l may be positioned adjacent the posterior gastric wall 142 of the stomach 112 (FIG. 7B).

FIG. 7B illustrates the tissue fasteners 230 of FIG. 7A each in a deployed configuration and positioned through a cutaway view of the stomach 112. In this instance, the T-tag 240 is illustrated with one end adjacent the anterior gastric wall 140 of the stomach 112, and the other end adjacent the posterior gastric wall 142 of the stomach 112. As described with reference to the embodiment of FIG. 5A, the rivet 237 is shown in the deployed position with the anterior rivet body 231 positioned adjacent the anterior gastric wall 140 and extending through the posterior gastric wall 142, and the posterior rivet base 233 positioned adjacent the posterior gastric wall 142 of the stomach 112. Similarly, in the deployed configuration, the multi-headed rivet 235 comprises the multi-headed anterior rivet body 239 adjacent the anterior gastric wall 140 of the stomach 112 and the posterior rivet base 233 adjacent the posterior gastric wall 142 of the stomach 112. Further, the magnet 244 is illustrated in a deployed configuration with a first magnet plate 242a positioned adjacent the anterior gastric wall 140 and a second magnet plate 242b positioned adjacent the posterior gastric wall 142, such that the stomach 112 may be compressed between the first and second magnet plates 242a, 242b as they attract each other with the stomach 112 between them. Additionally, FIG. 7B illustrates the magnet 246 in a deployed configuration such that magnet plates 242a-f are positioned adjacent the anterior gastric wall 140 and magnet plates 242g-l adjacent the posterior gastric wall 142, such that the stomach 112 may be compressed between the magnet plates 242a-f and magnet plates 242g-l, respectively as they attract each other. While the above are described as alternate embodiments of the one or the plurality of tissue fasteners 230, these are not limiting to the present disclosure and various other applicable tissue fasteners 230 may be applied.

FIG. 7C illustrates a top view the tissue fasteners 230 of FIG. 7A. In this instance, the T-tag 240 comprises a relatively linear portion positioned from the top view. Further, the anterior rivet body 231 of the rivet 237 and multi-headed anterior rivet body 239 of the adjustable rivet 235 are illustrated generally as discs from the top view. Further, a first of the magnet plates 242a is illustrated from the top view of the magnet 244 as illustrated in FIG. 7C. Additionally, magnet plates 242a-f of magnet 246 are illustrated in the top view of FIG. 7C.

FIG. 8 is a flow chart illustrating a method 300 for partially compartmentalizing a stomach 112. The method 300 will be described with reference to the partially compartmentalized stomach of FIG. 2A. At block 302, the method 300 first includes accessing the anterior gastric wall 140 and posterior gastric wall 142 that is between the restricted stomach portion 136 and the active stomach portion 134. At block 304, the method 300 further includes engaging the anterior gastric wall 140 and posterior gastric wall 142 towards each other in abutment at a location from the fundus 130 to adjacent the antrum 128. This step further includes the defining of the active stomach portion 134 and the restricted stomach portion 136.

At block 306, the method 300 then includes coupling the anterior gastric wall 140 and the posterior gastric wall 142 to secure the abutment, forming the septum 122 from the fundus 130 to adjacent the antrum 128 while retaining fluid communication between the active stomach portion 134 and the restricted stomach portion 136. In instances, the coupling includes a means for puncturing the tissue of the anterior gastric wall 140 and the posterior gastric wall 142 and securing with at least one of a plurality of tissue fasteners 230. In some instances, the coupling includes at least one of stapling, suturing and riveting the anterior and posterior gastric walls 140, 142. In other instances, this may include using a T-tag device, the T-tag device being absorbable or non-absorbable. In further instances, the coupling may include deploying a first magnet plate 242a adjacent the anterior gastric wall 140 and a second magnet plate 242b adjacent the posterior gastric wall 142 such that the anterior and posterior gastric walls 140, 142 couple when the first and second magnet plates 242a, 242b draw to one another. In this way, neither the anterior gastric wall 140 nor the posterior gastric wall 142 needs to be punctured to be coupled. In instances, the coupling of the anterior gastric wall 140 and posterior gastric wall 142 is configured such that it retains the pathway 138 below the septum 122 for fluid communication between the active stomach portion 134 and the restricted stomach portion 136. In instances, the septum 122 that is formed extends a majority of the length 141 from the fundus 130 to the antrum 128. In this way, the septum 122 extends the majority of the length 141 of the body cavity 132 such that the size of the pathway 138 for fluid communication, and therefore the amount of substance that has the potential to move from the active stomach portion 134 to the restricted stomach portion 136, is limited.

In instances, the method 300 of partially compartmentalizing the stomach 112 is completed through the use of the endoscopic gastric apparatus 200 including the first gastric wall engagement element 202 and the second gastric wall engagement element 204. While the following instance is described with reference to the endoscopic gastric apparatus 200 of FIGS. 3 and 4, the method 300 may be applied to other variations of the exemplary instance of the endoscopic gastric apparatus 200, such as the endoscopic gastric apparatus 200 of FIG. 5A.

In these instances, the accessing the anterior gastric wall 140 and the posterior gastric wall 142 step of block 302 further includes positioning of the first gastric wall engagement element 202 adjacent the anterior gastric wall 140 and positioning the second gastric wall engagement element 204 adjacent the posterior gastric wall 142. The engaging the anterior and posterior gastric walls 140, 142, respectively, of block 304 further includes engaging the first gastric wall engagement element 202 and the second gastric wall engagement element 204 towards each other. Additionally, the coupling step of block 306 may further include the deployment of the plurality of tissue fasteners 230 from one of the first and second gastric wall engagement elements 202, 204. In various embodiments, this step of coupling the anterior and posterior gastric walls 140, 142 may be repeated such that the septum 122 has two rows of the plurality of tissue fasteners 230.

Further, the method 300 may include removing the endoscopic gastric apparatus 200 from adjacent the anterior and posterior gastric walls 140, 142 of the stomach 112 without severing the anterior and posterior gastric walls 140, 142, and therefore, without severing the active stomach portion 134 and restricted stomach portion 136.

The invention of this application has been described above both generically and with regard to specific instances. It will be apparent to those skilled in the art that various modifications and variations can be made in the instances without departing from the scope of the disclosure. Thus, it is intended that the instances cover the modifications and variations of this invention provided they come within the scope of the appended claims and their equivalents.

1. An endoscopic gastric apparatus comprising:
an applier having a distal end and a proximal end,
the applier including two gastric wall engagement elements opposing one another and in cooperative arrangement for engaging an anterior gastric wall and a posterior gastric wall, respectively, into abutment and deploying one or a plurality of tissue fasteners, simultaneously or sequentially, operable for applying the one or the plurality of tissue fasteners to fix the anterior and posterior gastric walls together for the creation of a septum within a stomach defining an active stomach portion and a restricted stomach portion, wherein the septum allows for fluid communication between the active stomach portion and the restricted stomach portion through a pathway defined by the septum.

2. The endoscopic gastric apparatus of claim 1, wherein the applier is secured at the distal end with a support shaped and dimensioned for passage into an abdominal cavity.

3. The endoscopic gastric apparatus of claim 1, wherein the septum extends from a fundus to adjacent an antrum such that the pathway defined by the septum is adjacent the antrum.

4. The endoscopic gastric apparatus of claim 1, wherein each of the plurality of tissue fasteners is deployed sequentially.

5. The endoscopic gastric apparatus of claim 1, wherein each of the plurality of tissue fasteners is deployed simultaneously.

6. The endoscopic gastric apparatus of claim 1, wherein the pathway is sized as to allow for passage of substances between the restricted stomach portion and the active stomach portion.

7. The endoscopic gastric apparatus of claim 1, wherein the one or the plurality of tissue fasteners is at least one of a staple, suture, rivet, adjustable rivet, and opposing magnets.

8. The endoscopic gastric apparatus of claim 1, wherein a number of tissue fasteners deployed ranges from 1 to 40.

9. The endoscopic gastric apparatus of claim 1, wherein the endoscopic gastric apparatus comprises a first arced portion and a second arced portion at a proximal end of each of the two gastric wall engagement elements.

10. The endoscopic gastric apparatus of claim 9, wherein the first and second arced portions are configured to enclose a spacing when the endoscopic gastric apparatus is in a closed configuration, the spacing operable to form the pathway defined by the septum.

11. The endoscopic gastric apparatus of claim 1, wherein a first of the two gastric wall engagement elements comprises a cartridge housing the one or plurality of tissue fasteners.

12. The endoscopic gastric apparatus of claim 1, wherein a second of the two gastric wall engagement elements comprises of one or a plurality of tissue fastener receiving elements.

13. The endoscopic gastric apparatus of claim 12, wherein the one or the plurality of tissue fasteners comprises staples and the one or the plurality of tissue fastener receiving elements comprises staple pockets.

14. The endoscopic gastric apparatus of claim 1, wherein the two gastric wall engagement elements are configured to each have a length that is about between 200 mm and 250 mm.

15. The endoscopic gastric apparatus of claim 1, wherein the two gastric wall engagement elements are configured to each have a length that are about equal.

16.-30. (canceled)

31. A method for treating obesity, comprising:
using the endoscopic gastric apparatus of claim 1 to engage the anterior and posterior gastric walls towards each other in abutment at a location from a fundus to adjacent an antrum defining an active stomach portion and a restricted stomach portion; and
coupling the anterior and posterior gastric wall so as to secure the abutment and forming a septum while retaining fluid communication between the active stomach portion and the restricted stomach portion.

32. The method for treating obesity of claim 31, wherein the septum is formed from the fundus to adjacent the antrum.

33. The method for treating obesity of claim 31, wherein the fluid communication is retained through a pathway defined by the septum and a stomach wall.

34. The method for treating obesity of claim 31, wherein coupling the anterior and posterior gastric wall includes positioning one of the two gastric wall engagement elements adjacent the anterior gastric wall and the other of the two gastric wall engagement elements adjacent the posterior gastric wall and deploying the one or more plurality of tissue fasteners.

35. A method for treating diabetes, comprising:
using the endoscopic gastric apparatus of claim 1 to engage the anterior and posterior gastric walls towards each other in abutment at a location from a fundus to adjacent an antrum defining an active stomach portion and a restricted stomach portion; and
coupling the anterior and posterior gastric wall so as to secure the abutment and forming a septum from the fundus to adjacent the antrum while retaining fluid communication between the active stomach portion and the restricted stomach portion.

36. The method for treating diabetes of claim 35, wherein coupling the anterior and posterior gastric wall includes positioning one of the two gastric wall engagement elements adjacent the anterior gastric wall and the other of the two gastric wall engagement elements adjacent the posterior gastric wall and deploying the one or the plurality of tissue fasteners.
